SEQUENCE LISTING (6 sequences)

SEQ ID NO: 1 - DNA, 702 bp, homo sapiens
  CDS (1)..(702)
atgggatggagctgtatcatcctcttcttggtagcaacagctacaggtgtccactccgacatccagatgacccagagcccaagcagcctgagcgccagcgtgggtgacagagtgaccatcacctgtaaagcaagtcagaatattgacaaatacttaaactggtaccagcagaagccaggtaaggctccaaagctgctgatctacaatacaaacaatttgcaaacgggtgtgccaagcagattcagcggtagcggtagcggtaccgacttcaccttcaccatcagcagcctccagccagaggacatcgccacctactactgcttgcagcatataagtaggccgcgcacgttcggccaagggaccaaggtggaaatcaaacgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - DNA, 1413 bp, homo sapiens
  CDS (1)..(1413)
atgggatggagctgtatcatcctcttcttggtagcaacagctacaggtgtccactcccaggtccaactgcaggagagcggtccaggtcttgtgagacctagccagaccctgagcctgacctgcaccgtgtctggcttcaccttcaccgatttctacatgaactgggtgagacagccacctggacgaggtcttgagtggattggatttattagagacaaagctaaaggttacacaacagagtacaatccatctgtgaaggggagagtgacaatgctggtagacaccagcaagaaccagttcagcctgagactcagcagcgtgacagccgccgacaccgcggtctattattgtgcaagagagggccacactgctgctccttttgattactggggtcaaggcagcctcgtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 470 aa, homo sapiens
MGWSCIILFLVATATGVHSQVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 233 aa, homo sapiens
  PEPTIDE (1)..(233)
MGWSCIILFLVATATGVHSDIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 702 bp, homo sapiens
  CDS (1)..(702)
atgggctggtcctgcatcatactgttcctggtggctacggctacgggcgtgcattccgacatccaaatgacccaaagcccctcgtccctgtccgcgtccgtcggcgacagggttacaatcacgtgcaaagcgagccagaacatagacaagtacctgaactggtaccagcagaagccggggaaggcccccaagctgctgatatacaatactaataacctccagaccggcgtccccagccggttctccgggtccggttcgggcaccgacttcaccttcacaatcagttctctgcaaccggaggacatcgccacgtactattgcctgcaacatatcagtcggccccgaaccttcggccagggcaccaaggtggaaatcaaaaggaccgtagctgccccttccgtgttcatcttccccccctccgacgaacagctgaagtcgggcacagcgtccgtggtgtgcctcctgaacaacttctaccctcgggaggccaaagtgcagtggaaggtggacaacgccctgcagtctggcaactctcaggagtcggtgaccgagcaagacagtaaggatagcacctattccctatcctcgaccctgaccctgtcgaaggcggactacgagaagcacaaagtatacgcctgcgaggtgacccatcagggcctctcatcgcctgtgaccaaaagtttcaaccgcggcgagtgctag

SEQ ID NO: 6 - DNA, 1413 bp, homo sapiens
  CDS (1)..(1413)
atgggatggtcttgcattatcctttttctggtggcaacggccaccggggttcactcgcaagtccagctccaggagtccggcccaggcctggtgaggccgtcccagaccctgagtctgacctgcaccgtttccgggttcaccttcaccgatttctatatgaactgggtacggcagcccccagggcgcggtctggagtggatcggtttcattcgcgataaggccaaaggctacacaacagaatacaatcccagcgtcaaggggagggtgacgatgctggtagacacgagtaagaaccagttcagtttaagactctccagtgtgaccgccgctgacaccgccgtctattactgcgctcgcgaaggccacaccgccgcaccgttcgactactggggccagggctcgctggtgaccgtttccagtgcctccacaaagggtccttccgtctttcccctggcgccttcgagtaagagtaccagcggcggcacagcggccctgggctgcctggtcaaggactacttcccagagccggtcacggtgtcctggaattccggcgccctgacctccggggtgcacacattcccagcggtcctccagtcctccggtctgtactccctgtcctcggtggtgacagtgccctccagtagcctcggcacccagacatacatttgcaacgtgaaccataagccctccaatacgaaggtggataagaaggtggagcccaagtcctgtgataagacgcacacctgccccccttgccccgccccagagctgctgggagggcctagtgtgttccttttccccccgaagcccaaggacaccctgatgatttcccgcacccctgaagtcacgtgtgtcgtggtggacgtttcccatgaggaccctgaggtgaagtttaattggtatgtcgatggggtggaggttcacaatgcgaagacgaagcccagggaggagcaatacaactctacctaccgcgtcgtgtcagtcctgactgtcctgcaccaggactggctgaacgggaaggagtacaagtgcaaggtgtcgaacaaggctctgcccgccccgatcgagaagaccatcagcaaggccaagggccagcctcgagagccccaggtgtacacactgcccccgtccagggacgaactaaccaagaaccaggtgtccctgacctgcctggttaaaggattctacccatccgacatcgcagtcgagtgggagtccaacggccagccagaaaacaattataaaacgactcctcccgtcctggacagcgacggctcattctttctgtactcgaagctgacagtggacaagagccgctggcagcaggggaacgtgttctcctgcagtgtcatgcacgaggccctgcataaccactacacacagaagtccctgagcctgtcgcccggaaagtga

(19) United States
(12) Patent Application Publication
(10) Pub. No.: US 2009/0220520 A1
(43) Pub. Date: Sep. 3, 2009

(54) Recombinant method for the production of a monoclonal antibody to CD52 for the treatment of chronic lymphocytic leukemia

(76) Inventor: Villoo Morawala Patell, Bangalore (IN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/914,752

(22) PCT Filed: May 24, 2006
(86) PCT No.: PCT/IB06/01356

§ 371 Date: Nov. 18, 2007

(30) Foreign Application Priority Data
May 24, 2005 (IN) 625/CHE/2005

(51) Int. Cl. A61K 39/395; C07K 16/28; C12P 21/04

(52) U.S. Cl. 424/143.1; 530/387.1; 435/69.6

(57) ABSTRACT

The present invention relates to the recombinant method used for the production of soluble form monoclonal antibody that binds to CD52. The procedure describes the de novo synthesis of the nucleic acid sequence encoding anti-CD 52, transformation of the constructed nucleic acid sequences into competent bacteria and the sub-cloning of the same into mammalian expression vectors for expression of the desired protein. DNA constructs comprising the control elements associated with the gene of interest has been disclosed. The nucleic acid sequence of interest has been codon optimized to permit expression in the suitable mammalian host cells.

### FIELD OF THE INVENTION

The present invention relates to the recombinant method used for the production of soluble form monoclonal antibody that binds to CD52. The procedure describes the de novo synthesis of the nucleic acid sequence encoding anti-CD 52, transformation of the constructed nucleic acid sequences into competent bacteria and the sub-cloning of the same into mammalian expression vectors for expression of the desired protein. DNA constructs comprising the control elements associated with the gene of interest has been disclosed. The nucleic acid sequence of interest has been codon optimized to permit expression in the suitable mammalian host cells.

### BACKGROUND OF THE INVENTION

Antibodies are a part of our immune system. When an antigen (such as a foreign protein in a germ) enters the body, the body makes natural antibodies to fight against it. These antibodies attach to the antigen and mark it for destruction by our immune system. Antibodies, or immunoglobulins, comprise two heavy chains linked together by disulphide bonds and two light chains, each light chain being linked to a respective heavy chain by disulphide bonds. Each heavy chain has at one end a variable domain (VH) followed by a number of constant domains (CH1, 2, 3). Each light chain has a variable domain (VL) at one end and a constant domain (CL) at its other end, the light chain variable domain of the heavy chain (VH) and the light chain constant domain being aligned with the first constant domain of the heavy chain. The constant domains in the light and heavy chains (Fc) are not involved directly in binding the antibody to antigen.

The variable domains of each pair of light and heavy chains (Fab) form the antigen-binding site. The domains on the light and heavy chains have the same general structure and each domain comprises four framework regions, whose sequences are relatively conserved, connected by three complementarity regions (CDRs). The four framework regions largely adopt a beta-sheet conformation and the CDRs form loops connecting, and in some cases forming part of, the beta-sheet structure. The CDRs are held in close proximity by the framework regions and, with the CDRs from the other domain, contribute to the formation of the antigen-binding site.

A major step forward occurred in 1975 when Kohler and Milstein (Nature, 1975, 256, 495-497) reported the successful fusion of spleen cells from mice immunized with an antigen with cells of a murine myeloma line. The resulting hybrid cells, termed hybridomas, have the properties of antibody production derived from spleen cells and of continuous growth derived from the myeloma cells. Each hybridoma synthesizes and secretes a single antibody to a particular determinant of the original antigen. To ensure that all cells in a culture are identical, i.e. that they contain the genetic information required for the synthesis of a unique antibody species, the hybridomas resulting from cell fusion are cloned and subcloned. In this way, the cloned hybridomas produce homogeneous or monoclonal antibodies. The immortality of the cell line assures that an unlimited supply of a homogeneous, well-characterized antibody is available for use in a variety of applications including in particular diagnosis and immunotherapy of pathological disorders. Unfortunately, monoclonal antibody developed were not useful in a clinical setting can be severely hampered by the development of human anti-mouse antibodies, which may interfere with therapy or cause allergic or immune complex hypersensitivity.

To generate sufficient quantities of antibody for full clinical use it is desirable to employ an efficient recombinant expression system. Since myeloma cells represent a natural host specialized for antibody production and secretion, cell lines derived from these have been used for the expression of recombinant antibodies. Often, complex vector design, based around immunoglobulin gene regulatory elements, is required, and final expression levels have been reported which are highly variable (Winter et al, Nature, 1988, 332, 323-327; Weidle et al, Gene, 1987, 60, 205-216; Nakatani et al, Bio/Technology, 1989, 7, 805-810; and Gillies et al, Bio/Technology, 1989, 7, 799-804). An alternative mammalian expression system is that offered by the use of Chinese hamster ovary (CHO) cells. The use of these cells has enabled the production of large quantities of several therapeutic proteins for research and clinical use (Kaufman et al, Mol. Cell. Biol, 1985, 5, 1750-1759; and Zettlmeissl et al, Bio/Technology, 1987, 5, 720-725). There are, however, very few instances of the use of these cells for the expression of antibodies and the levels of expression of murine antibodies reported to date are low of the order of 0.01-0.1. μ/ml (Weidle et al, Gene, 1987, 51, 21-29; and Feys et al, Int. J. Cancer, 1988, 2, 26-27).

CD52 is strongly expressed on virtually all human lymphocytes and monocytes, but it is absent from other blood cells including the hemopoietic stem cells, the antigen being described by Hale et al, Blood 1983, 62, 873-882. A mouse monoclonal antibody was developed YTH66.9 which is specific to CD52 and later a rat monoclonal antibody to CD52 YTH 34.5HL. The use of these antibodies in clinical setting was limited due to antiglobulin response.

CD52 is expressed on the surface of normal and malignant B and T lymphocytes, NK cells, monocytes, macrophages, and tissues of the male reproductive system. The present invention relates to a method wherein an antibody that binds to CD25 is indicated for the treatment of B-cell chronic lymphocytic leukemia (B-CLL). The antibody can also be used for treating patients who have been treated with alkylating agents and who have failed fludarabine therapy. The antibody in the invention is an IgG1 kappa with human variable framework and constant regions, and complementarity-determining regions from a murine (rat) monoclonal antibody. The antibody has an approximate molecular weight of 150 Kd.

The present invention would permit the production of anti-CD52 in mammalian expression vector system by the incorporation of the recombinant vectors comprising the nucleic acid sequences encoding the polypeptides possessing the activity of anti-CD52.

The anti-CD52 antibody will be used to treat B-cell chronic lymphocytic leukemia also known as B-CLL. The antibody will also be used to treat patients that have already received and/or have not responded to other cancer chemotherapy drugs (e.g., alkylating agents, fludarabine). The antibody will also find use in, non-hodgkin lymphoma, treatment of some autoimmune diseases, kidney graft patients.

In a further aspect the anti-CD52 antibody will be used for treating humans with cancers, particularly lymphomas, or for immunosuppression purposes.

### DESCRIPTION OF FIGURES

FIG. 1. Pair-wise alignment of the non-optimized and codon optimized versions of the DNA nucleotide sequence encoding the light chain of the anti-CD52 antibody.

FIG. 2. Pair-wise alignment of the non-optimized and codon optimized versions of the DNA nucleotide sequence encoding the heavy chain of the anti-CD52 antibody.

FIG. 3. Alignment of the light chain constant domains of anti-CD20 antibody (Rituximab) and anti-CD52 antibody (Campath)

FIG. 4. Alignment of the heavy chain constant domains of anti-CD20 antibody (Rituximab) and anti-CD52 antibody (Campath).

FIG. 5. Restriction digestion of pBSKII/ALZ-VLC (lane 3) and the pBSKII/RTX-LC (lane 1) clones with BglII BsiWI. Lane 2-1 kb DNA ladder.

FIG. 6. Restriction digestion of the pBSK I/ALZ-LC clones with BglII and EcoRI for identification of the transformants containing the insert. For all the clones tested a fall-out fragment corresponding to the size of the ALZ-LC antibody fragment was observed.

FIG. 7. Restriction digestion of the pBSKII/ALZ-LC clones with BamHI for distinguishing the clones containing ALZ-LC insert from the clones containing the RTX-LC.

FIG. 8. Restriction digestion of the pBSKII/ALZ-VHC full length clones to confirm the presence of the insert.

FIG. 9. Sequence alignment of the anti-CD20 antibody heavy chain (RTX-HC) with the anti-CD52 antibody heavy chain (ALZ-HC) clone subjected to site directed mutagenesis. The CA to TT change is shown highlighted in blue.

FIG. 10. Restriction digested and gel purified ALZ-LC and pCAIN for ligation reaction.

FIG. 11.: Restriction digestion of the pCAIN/ALZ-LC clones with BglII and EcoRI for identification of the transformants containing the insert. For all the clones tested a fall-out fragment corresponding to the size of the ALZ-LC antibody fragment was observed (˜700 bp).

FIG. 12. Restriction digested and gel purified ALZ-HC and pCAID for ligation reaction.

FIG. 13. Restriction digestion of the pCAID/ALZ-HC clones with BamHI and EcoRI for identification of the transformants containing the insert. For all the clones tested a fall-out fragment corresponding to the size of the ALZ-HC antibody fragment was observed (˜1420 bp).

FIG. 14. The clone b of pCAID/ALZ-HC was confirmed by DNA nucleotide sequencing. Three different primers (camp246, 523 and CHC845) were used for the sequencing reaction. The whole sequence was found to match to the template sequence (C2).

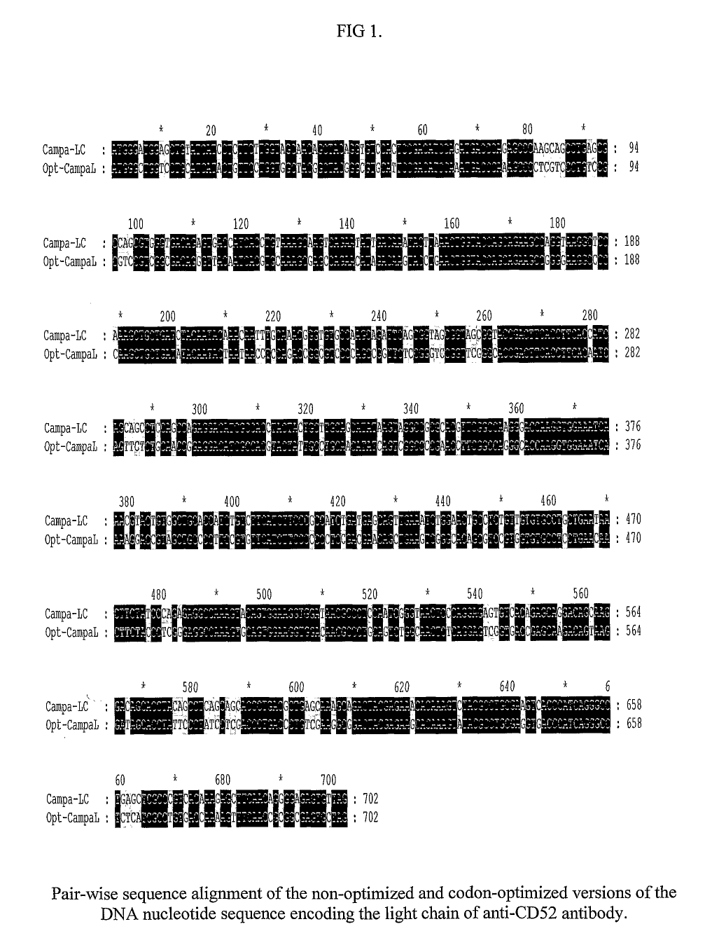

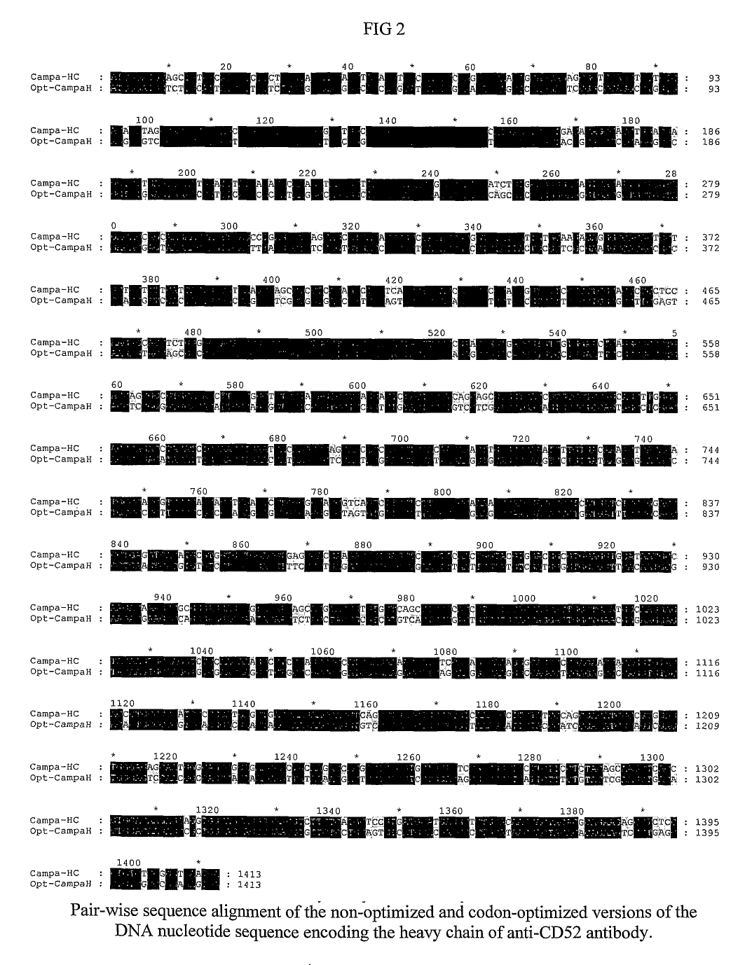

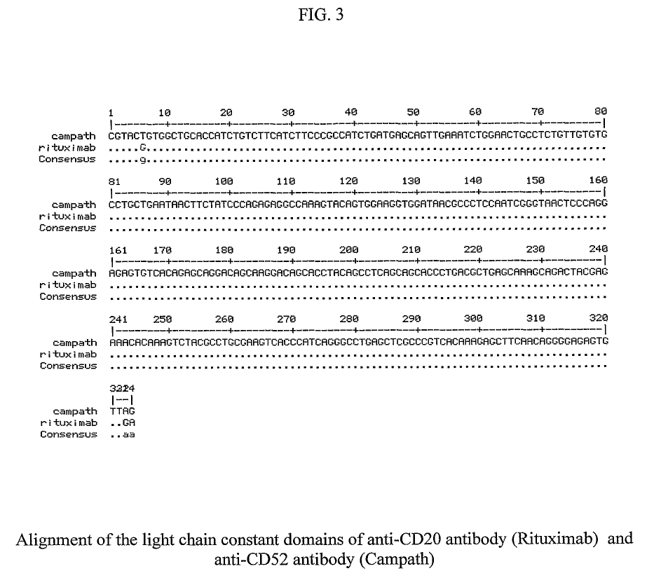

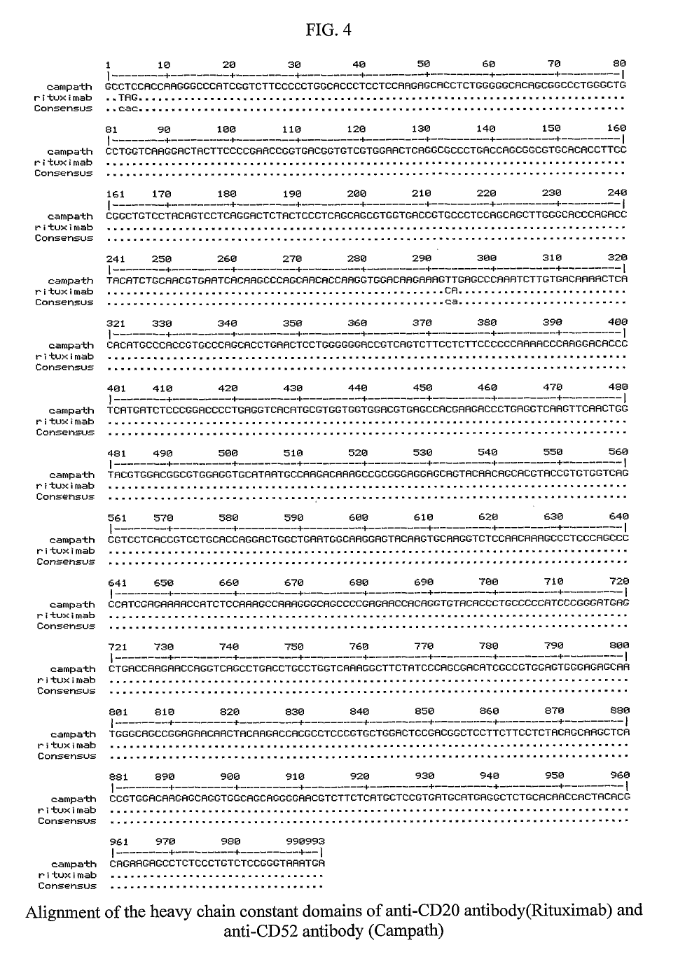

### SEQUENCE LISTINGS

SEQ ID 1. Nucleotide sequence of the anti-CD52 antibody of the light chain

SEQ ID 2. Nucleotide sequence of the anti-CD52 antibody of the heavy chain

SEQ ID 3. Amino acid sequence of the anti-CD52 antibody peptide

SEQ ID 4. Amino acid sequence of the anti-CD52 antibody peptide

SEQ ID 5. codon optimized sequence of the anti-CD52 antibody of the light chain

SEQ ID 6. codon optimized sequence of the anti-CD52 antibody of the heavy chain

### SUMMARY OF THE INVENTION

The present invention is directed to the transformation of nucleic acid sequence encoding the polypeptide anti-CD52 into competent bacteria and the subcloning of the same into mammalian expression vectors more preferably into CHO cells for the stable expression of the said monoclonal antibody.

According to an aspect of the invention there is provided the nucleic acid sequences encoding the heavy and the light chains of the anti-CD 52 molecule. According to an further aspect of the invention there is provided the corresponding amino-acid sequence encoded by the nucleic acid sequences.

A particular aspect of the invention relates to the de novo synthesis of the variable regions of the heavy and the light chain of the anti-CD 52 molecule. Further disclosed is the construction of the vector constructs with the nucleic acid sequence of interest, transformation of the vector constructs into competent bacteria and subcloning of the anti-CD52 chains into mammalian expression vectors.

### DETAILED DESCRIPTION OF THE INVENTION

The procedure outlined below is suitable for the production of bioactive, recombinant soluble anti-CD52 antibody.

### Example 1

### De Novo Synthesis of the cDNA of Anti-CD52 Antibody would Include the Following Components

A Kozak consensus sequence (GCCACC) 3 followed by an initiation codon (ATG)
Three “protecting” nucleotides at the 5′ and 3′ end of the cDNA
Suitable restriction sites at the 5′ and 3′ end of the cDNA to clone into the expression vector.

Nucleotide sequence encoding the light chain of anti-CD52 antibody has been represented in SEQ ID 1.

Nucleotide sequence encoding the heavy chain of anti-CD52 antibody has been depicted in SEQ ID 2.

The codons in the coding DNA sequence of the heavy chain of anti-CD52 antibody that have been altered as part of the codon-optimization process to ensure optimal recombinant protein expression in mammalian cell lines such as CHO K1 and HEK 293 The respective codon optimized sequences have been represented in SEQ ID 4 and 5.

### Example 2

### Choice of the Expression System

The design of the mammalian expression vector for the expression of recombinant anti-CD52 antibody can be based on one of the commercially available vectors (eg: pcDNA or pIRES from Invitrogen or BD Biosciences respectively), modified to include the following features:
multiple cloning site for insertion of the cDNA encoding the light chain and heavy chain of anti-CD52 antibody along with the natural signal peptide. The light chain and heavy chain of anti-CD52 antibody will be cloned into two separate plasmid DNA vector with different selection marker.
The design of the vector can also accommodate an independent (bi-cistronic) IRES-mediated co-expression of the selection marker for both the light chain and the heavy chain.
The design of the expression vector can also accommodate an independent (bi-cistronic) IRES-mediated co-expression of both the light chain and heavy chain of anti-CD52 antibody along with the natural signal peptide.

### Example 3

### De Novo Synthesis of the Variable Domains of Anti-CD52 Antibody

The light and heavy chain variable domains of anti-CD52 antibody were given for de novo synthesis to Epoch labs, USA. The anti-CD52 antibody chains were not synthesized with the constant domains; instead the kappa and IgG1 constant domains were excised from the anti-CD20 antibody chains and ligated with the variable domains of anti-CD52 antibody to generate full-length antibody chains.

The constant domains of the anti-CD52 antibody and anti-CD20 antibody are very similar. While the kappa constant domain of anti-CD52 antibody is 100% homologous to anti-CD20 antibody, the heavy chain constant domain differs by 2 nucleotides. The two-nucleotide change leads to a valine to alanine change at position 240.

### Example 4

### Construction of the Full Length Heavy and Light Chains of the Anti-CD52 Antibody

The variable domains that were synthesized de novo were cloned into pBSK vectors containing the full-length anti-CD20 antibody heavy and light chains. The variable domains of the anti-CD20 antibody were exchanged with the variable domains of the anti-CD52 antibody to yield the full-length anti-CD52 antibody fragments.

The variable domains of the anti-CD52 antibody were obtained as cloned fragments in pBSKII and the resultant construct referred to pBSKII/ALZ-VLC. The DNA was transformed into DHI10b E. coli cells and plated onto LB agar plates containing ampicillin. A colony from the plate was inoculated in liquid medium and a DNA mini prep was carried out. The sequence of the two variable domains was confirmed by sequencing.

### Construction of the Full-Length Kappa Light Chain of Anti-CD52 Antibody.

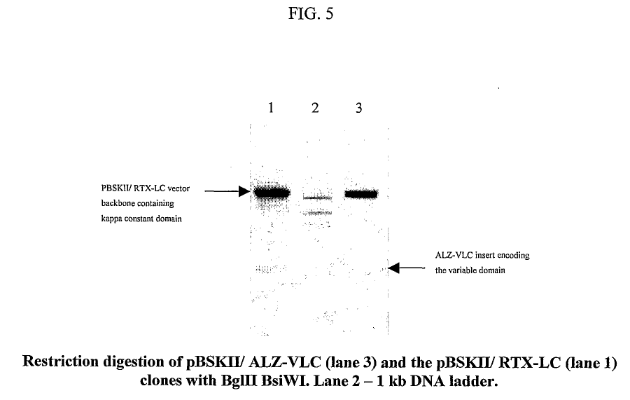

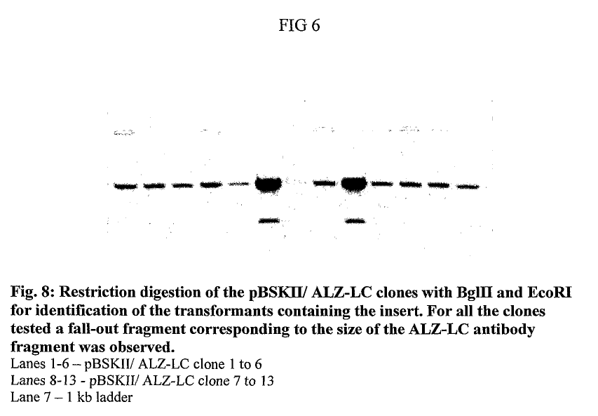

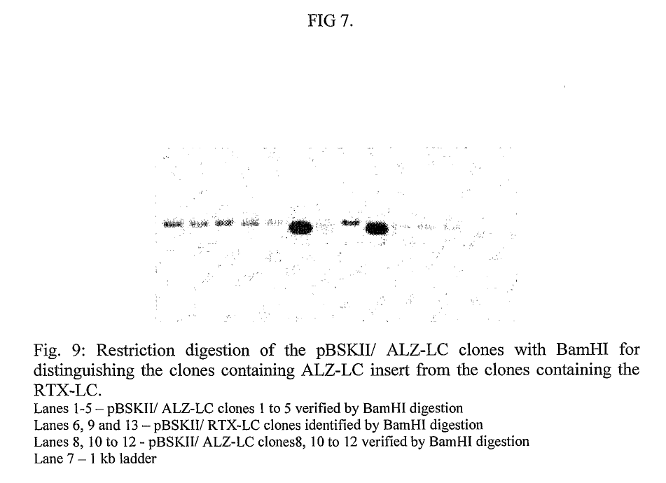

The pBSKII/ALZ-VLC and the pBSKII/RTX-LC (construct expressing anti-CD20 antibody light chain) clones were digested with BglII and BsiWI restriction enzymes (FIG. 5). The insert of size 394 bps from the former and the vector+kappa constant domain from the latter were gel purified. The vector and insert were ligated to yield the full-length anti-CD52 antibody kappa light chain. The colonies obtained on transformation of heat shock competent DH10 cells were inoculated in LB Amp medium, and a mini prep of the DNA was done. The clones were checked by restriction digestion for the presence of the full-length light chain of the anti-CD52 antibody (FIG. 6). The clones found positive by restriction digestion were also confirmed by sequencing The pBSKII/ALZ-LC clones were also digested with BamHI restriction enzyme for distinguishing the clones containing ALZ-LC insert from the clones containing the RTX-LC. This was essential because the pBSKII/RTX-LC was used as the vector backbone and the RTX-LC is of the same size as that of the ALZ-LC. The restriction enzyme pattern differs between the two light chains. While pBSKII/ALZ-LC clones when digested with BamHI will only linearize the pBSKII/RTX-LC clones will give a fall-out fragment of 700 bps (FIG. 7). The clone number 6, 9 and 13 were RTX-HC clones.

### Construction of the Full-Length IgG1 Heavy Chain of Anti-CD52 Antibody

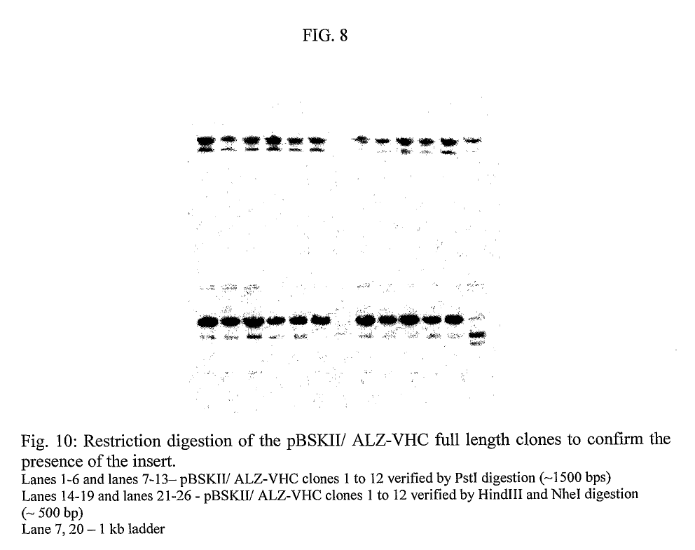

The construct pBSKII/ALZ-VHC harboring the anti-CD52 antibody variable heavy chain and the construct pBSKII/RTX-HC clones harboring the anti-CD20 antibody heavy chain were digested with HindIII and NheI restriction enzymes. The insert of size 426 bps from the former and the vector+IgG1 constant domain from the latter were gel purified. The vector and insert were ligated to yield the full-length anti-CD52 antibody IgG1 heavy chain. The colonies obtained on transformation of heat shock competent DH10 cells were inoculated in LB Amp medium, a mini prep of the DNA was done and the clones were checked by restriction digestion for the presence of the full length heavy chain of the anti-CD52 antibody (FIG. 8). The clones found positive by restriction digestion were also confirmed by sequencing.

Site Directed Mutagenesis of the pBSKII/ALZ-HC Clone

The constant domain of the anti-CD52 antibody heavy chain fragment differs from the anti-CD20 antibody heavy chain fragment in the constant domain by two nucleotides. A clone containing the variable domain of the anti-CD52 antibody spliced with the constant domain of the anti-CD20 antibody heavy chain after sequence verification was subjected to site directed mutagenesis.

### Material and Methods:

### Enzymes and Reagents:

Pfu high fidelity Taq Polymerase (Stratagene)
DpnI restriction enzyme (Stratagene)

### DNA Template

### PBSKII/ALZ-HC-clone (a)

### Primers

All synthetic oligonucleotides were synthesized by SIGMA. The synthesized oligonucleotides were purified twice on an HPLC by Sigma and transferred to the Avesthagen. The oligonucleotides listed in the table below were used for the PCR reactions.

!PRIMER? SEQUENCE

| | |
|---|---|
| Campath-mut-sense | 5′ AAGGTGGACAAGAAAGTTGAGCCCAAATCTTGT 3′ |
| Campath-mut-antisense | 5′ ACAAGATTTGGGCTCAACTTTCTTGTCCACCTT 3′ |

### Mutant Strand Synthesis Reaction (Thermal Cycling):

The following components/reagents were added to a sterile 0.2 ml PCR tube in the order indicated below. The final volume of all PCR reactions was 50 μl.

| Step | Final conc. | | PCR #1 | |
|---|---|---|---|---|
| Water | — | | 32.0 | μl |
| 10xPCR buffer | 1x | | 5.0 | μl |
| dNTP each | | | 1.0 | μl |
| Forw. primer | 125 | ng | 5.0 | μl |
| Backw. primer | 125 | ng | 5.0 | μl |
| DNA | | | 2 | μl |
| | | | (25 | ng) |
| Final volume | 50 | μl | 50 | μl |
| PfuTurbo DNA polymerase | 0.05 | U | 1.0 | μl |

### Cycling Parameters for the Quick Change Site-Directed Mutagenesis Method:

| | |
|---|---|
| Step 1 (1 cycle) | 95° C./30 seconds |
| Step 2 | 95° C./30 secs |
| Step 3 | 55° C./30 secs |
| Step 4 | 68° C./7 minute |
| Step 5 go to step 2 and repeat | |
| the steps 2-4 for 16 times | |

### Dpn I Digestion of the Amplification Products:

1 μl of Dpn I (10 U/μl) was added to the amplification reaction. the reaction mixture. The mixture was gently and thoroughly mixed, spun down and incubated at 37° C. for 1 hour.

### Transformation of DH10b Competent Cells:

The transformation was carried out as recommended by the manufacturer (Stratagene). 4 μl of the PCR reaction was used for transformation.

### Results:

Sequence alignment of the anti-CD20 antibody heavy chain (RTX-HC) with the anti-CD52 antibody heavy chain (ALZ-HC) clone subjected to site directed mutagenesis. The CA to TT change is shown highlighted in blue.

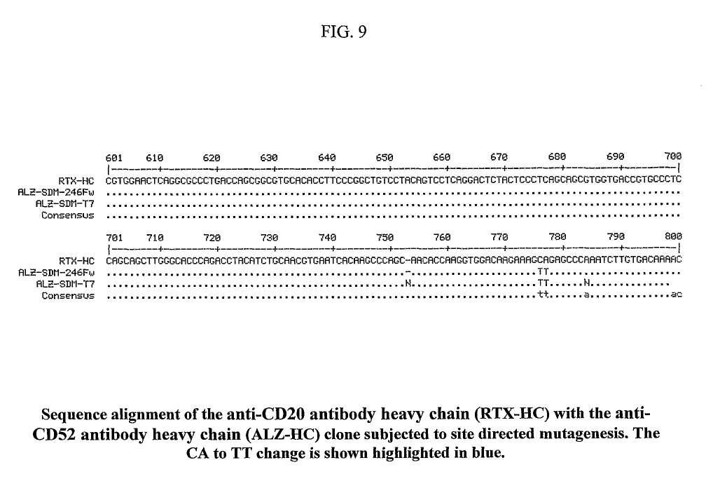

Sequence alignment of the anti-CD20 antibody heavy chain (RTX-HC) with the anti-CD52 antibody heavy chain (ALZ-HC) clone subjected to site directed mutagenesis. It has been represented in FIG. 9

### Sub-Cloning of the Anti-CD52 Antibody Chains in Mammalian Expression Vectors

The full-length anti-CD52 antibody heavy chain antibody light and heavy chains were cloned sub-cloned into mammalian expression vectors pCAIN and pCAID respectively.

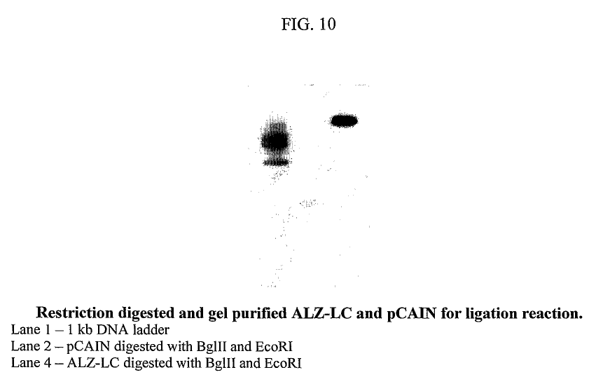

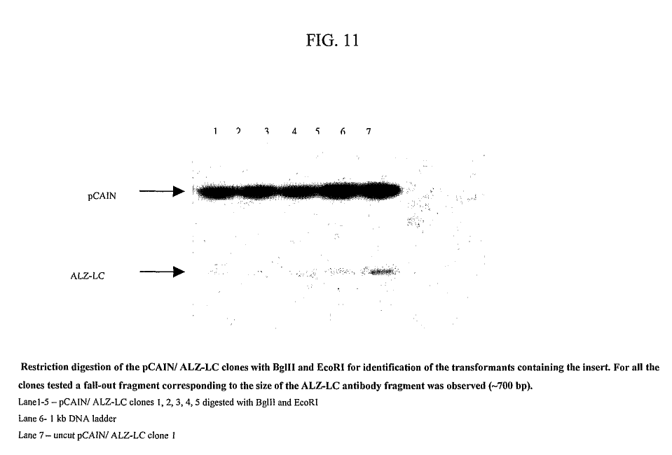

The pBSKII/ALZ-LC clone and the pCAIN vector were digested with BglII and EcoRI and resultant construct is referred to as pCAIN/ALZ-LC. The insert (700 bp) from the former and the vector backbone from the latter were gel purified and ligated (FIG. 10). The ligation mix was transformed into heat shock competent DH10b cells and plated onto LB agar plates containing ampicillin as the selection antibiotic. A few transformants were picked and a DNA mini prep was carried out. The clones were checked by restriction enzyme digestion (FIG. 11).

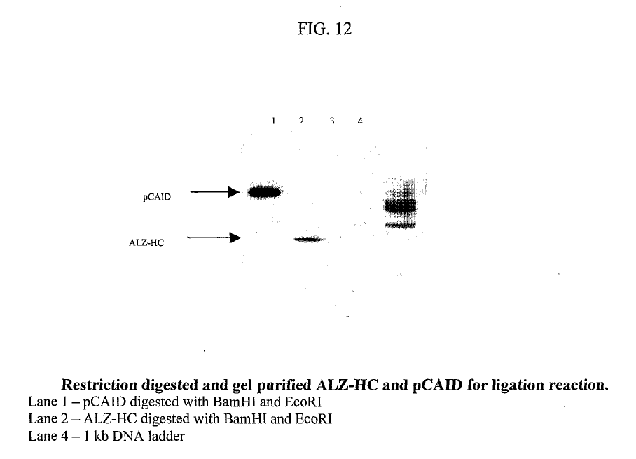

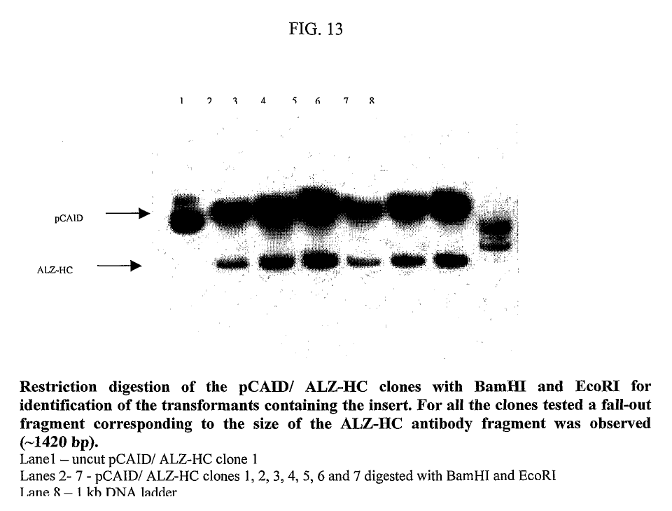

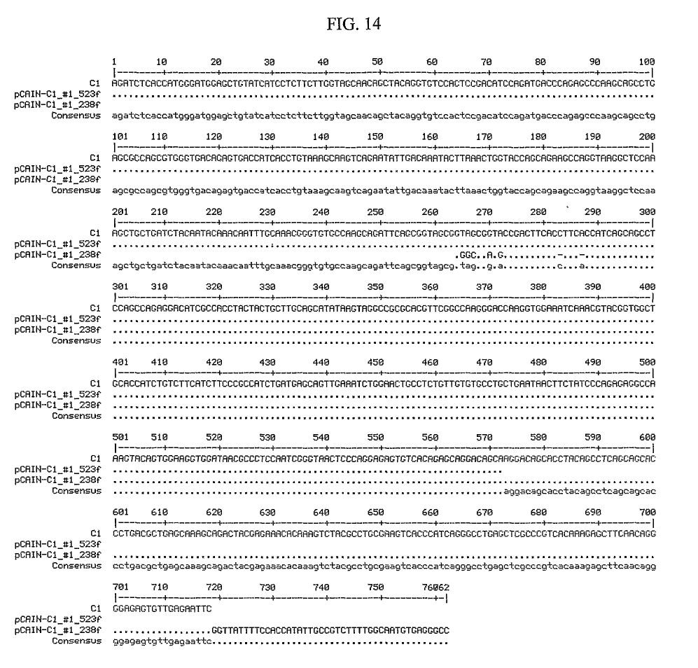

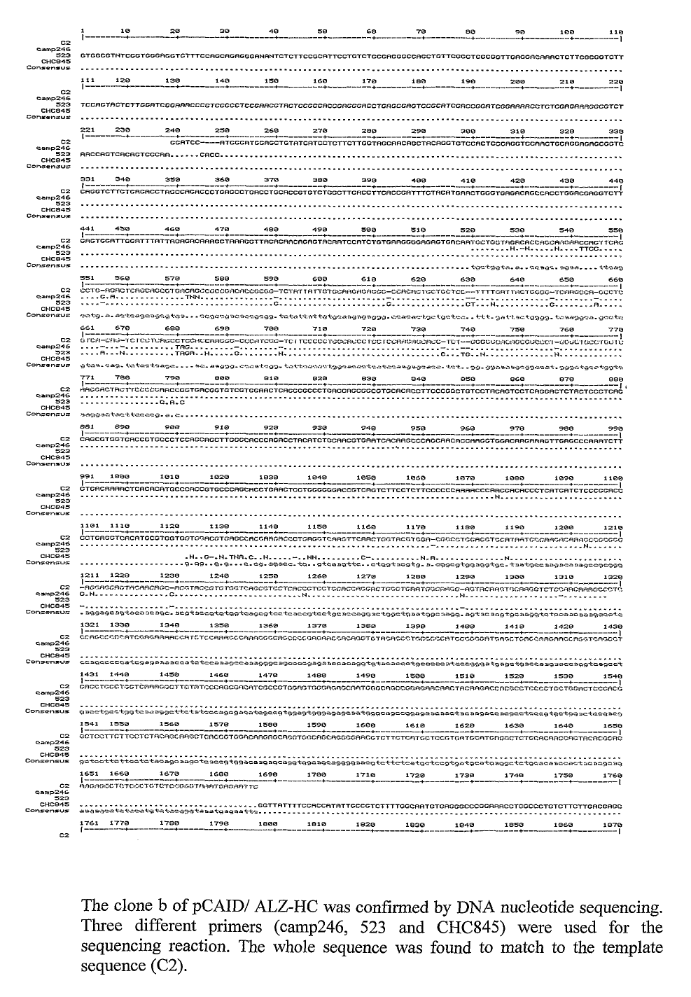

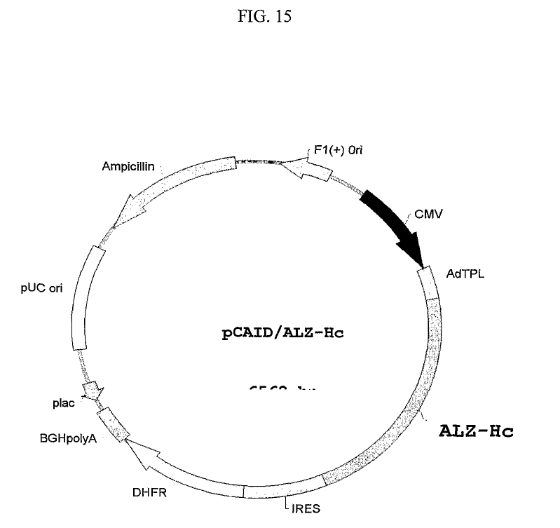

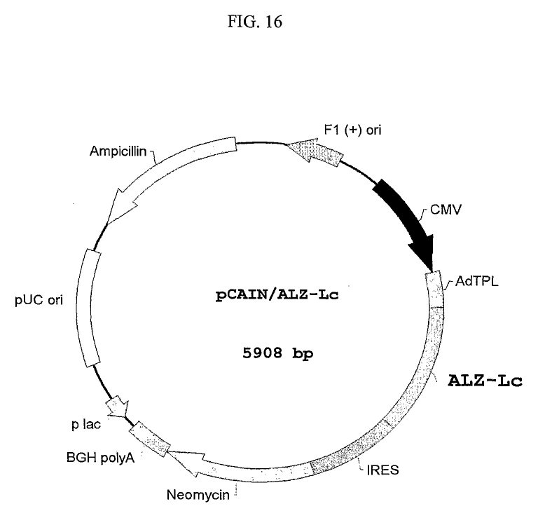

The pBSKII/ALZ-HC-SDM clone (1.4) and the pCAID vector were digested with BamHI and EcoRI. The resultant construct is referred to as pCAID/ALZ-HC. The insert (1429 bp) from the former and the vector backbone from the latter were gel purified and ligated (FIG. 12). The ligation mix was transformed into heat shock competent DH10b cells and plated onto LB agar plates containing ampicillin as the selection antibiotic. A few transformants were picked and a DNA mini prep was carried out. The clones were checked by restriction enzyme digestion (FIG. 13).

### DNA Sequencing and Analysis:

The final clones of ALZ-HC and ALZ-LC cloned into pCAID and pCAIN mammalian expression vectors respectively were sequenced and their sequence accuracy confirmed. The alignments of the sequence analysis have been represented.

The clone b of pCAID/ALZ-HC was confirmed by DNA nucleotide sequencing. Three different primers (camp246, 523 and CHC845) were used for the sequencing reaction. The whole sequence was found to match to the template sequence (C2).

### 6. Purification of Anti-CD52 Antibody:

Anti-CD52 antibody is a humanized antibody, which is secreted into the cell supernatant. Subsequent to the establishment of a contaminant-free cell culture system as per the guidelines of the regulatory agencies, that over-expresses the desired recombinant protein, the purification of anti-CD52 antibody protein can be done using a series of steps involving dialysis-filtration and column chromatography procedures involving affinity chromatography. The eluted antibody will be recovered to maximize in vivo activity.

### 7. Assays for In Vitro and In Vivo Activity of Anti-CD52 Antibody:

Bioassays for detecting in vitro binding activity of the anti-CD52 antibody will be done using:
Anti-CD52 antibody ELISA assay.
Complement mediated lysis of B cell lymphocytic leukemia cells (Karpas 422 cells)
The anti-CD52 antibody in vivo efficacy studies have been carried out in human patients with B-cell chronic lymphocytic leukemia (B-CLL) who have been previously treated with alkylating agents and had failed treatment with fludarabine based on currently available data.

### Optimisation of Purification Procedures:

Subsequent to the establishment of reproducible bioactivity in accordance with the recommended functional/binding assays mentioned above, efforts will be made to optimize the purification procedures. The purification strategies will aim at process economics, speed to market, scalability, reproducibility, and maximum purity of the product with functional stability and structural integrity as the major objectives. To this effect, a combinatorial approach with both filtration (normal and tangential flow filtration) and chromatography would be explored. The process qualification requirements and acceptance criteria studies will be conducted on 3 batches. Accordingly, the current invention envisages the following steps in the purification process:
a. Initial clarification using COHC/AlHC/0.45μ depth filters
b. Concentration using Pellicon XL Biomax 50 kDa cut-off filter based on tangential flow filtration
c. Chromo step—I: Affinity chromatography using Prosep VA Ultra for serum based (2% fetal calf serum [FCS])/and Prosep VA for serum free culture supernatants.
d. Chromo step —II: Strong cation exchanger such as SP Sepharose
e. Chromo step —III: Flow through based strong anion exchanger such as Cellufine Q (a cellulose based medium) for the removal of host cell proteins and nucleic acids.
f. Virus removal using size exclusion filtration and leached protein A using Cellufine sulfate
g. Sterile filtration
h. Endotoxin removal using either Remtox/Cellufine ET chromatography
h. Formulation

1. A process of preparing in vivo biologically active anti-CD 52 monoclonal antibody comprising the steps:
de novo synthesis of light and heavy chains of the anti-CD52 monoclonal antibody;
construction of full-length kappa light chain of the anti-CD 52 antibody;
construction of full length IgG1 heavy chain of the anti-CD 52 antibody;
construction of a vector comprising the nucleic acid sequences encoding the light and the heavy polypeptide chains of the anti-CD52 molecule; and
subcloning of the anti-CD 52 antibody chains in a mammalian expression vector for production of the biologically active antibody molecule.

2. The method according to claim 1, wherein the nucleotide sequence encoding the light chain of the anti-CD52 antibody has been represented in SEQ ID NO:1.

3. The method according to claim 1, wherein the nucleotide sequence encoding the heavy chain of the anti-CD 52 antibody has been represented in SEQ ID NO:2.

4. The method according to claim 1, wherein the amino acid sequence of the light chain of the anti-CD52 antibody has been depicted in SEQ ID NO:3.

5. The method according to claim 1, wherein the amino acid sequence of the heavy chain of the anti-CD 52 antibody has been depicted in SEQ ID NO:4.

6. The method according to claim 1, wherein the vector comprising the nucleic acid fragment encoding the heavy chain of the anti-CD52 is subjected to site-directed mutagenesis.

7. The method according to claim 1, wherein the full-length anti-CD52 heavy and light chain are subcloned into mammalian vectors pCAIN and pCAID respectively.

8. A method of preparation of an in vivo biologically active anti-CD52 monoclonal antibody comprising the steps of transforming a host cell with a vector construct of FIG. 15 or 16 and isolating said product from the host cell or the medium of its growth.

9. A pharmaceutical composition comprising a therapeutically effective amount of anti-CD 52 antibody and a pharmaceutically acceptable diluent, adjuvant or carrier, wherein said antibody is purified from mammalian cells grown in culture.
